SEQUENCE LISTING (4 sequences)

SEQ ID NO: 1 - RNA, 22 nt, synthetic construct
  source 1..22: mol_type = other RNA
gaatgttgctcggtgaacccct

SEQ ID NO: 2 - DNA, 22 bp, synthetic construct
  source 1..22: mol_type = other DNA
gaatgttgctcggtgaacccct

SEQ ID NO: 3 - DNA, 20 bp, synthetic construct
  source 1..20: mol_type = other DNA
ggggctcagtttcctcatct

SEQ ID NO: 4 - DNA, 20 bp, synthetic construct
  source 1..20: mol_type = other DNA
tagagacttgcagtggggtg

(19) United States
(12) Patent Application Publication
(10) Pub. No.: US 2026/0209854 A1
(43) Pub. Date: Jul. 23, 2026

(54) Method for Diagnosing Hearing Loss Using Changes in miR-409-3p Expression and Method for Screening Therapeutic Agents for Hearing Loss

(71) Applicant: AJOU UNIVERSITY INDUSTRY-ACADEMIC COOPERATION FOUNDATION, Gyeonggi-do (KR)

(72) Inventors: Jungho Ha, Gyeonggi-do (KR); Yun-Hoon Choung, Seoul (KR); SeongJun Choi, Seoul (KR); Youngsun Kim, Gyeonggi-do (KR); SeongJun So, Gyeonggi-do (KR)

(21) Appl. No.: 19/451,176

(22) Filed: Jan. 16, 2026

(30) Foreign Application Priority Data
Jan. 17, 2025 (KR) 10-2025-0007499

(51) Int. Cl. C12Q 1/6883

(52) U.S. Cl. CPC C12Q 1/6883; C12Q 2600/106; C12Q 2600/158; C12Q 2600/178

(57) ABSTRACT

The present invention relates to a method for diagnosing hearing loss by measuring the expression level of miR-409-3p, screening a therapeutic agent for hearing loss, and treating hearing loss by administering miR-409-3p. More specifically, the present invention relates to a composition for diagnosing hearing loss, comprising an agent for measuring an expression level of miR-409-3p as an active ingredient; a method for providing information for diagnosing hearing loss; a method for screening a therapeutic agent for hearing loss; and a pharmaceutical composition for preventing or treating hearing loss comprising miR-409-3p as an active ingredient.

### CROSS-REFERENCE TO RELATED APPLICATION

This application claims the benefit of Korean Patent Application No. 10-2025-0007499, filed on Jan. 17, 2025, the entire contents of which is incorporated by reference herein.

### INCORPORATION BY REFERENCE OF SEQUENCE LISTING FILE

This application incorporates by reference the material contained in the Sequence Listing XML file being submitted concurrently herewith: File name: SOP117343US_Sequence_Listing.xml; created Jan. 16, 2026, 4,499 bytes in size.

### TECHNICAL FIELD

The present invention relates to a technology for diagnosing hearing loss by measuring the expression level of miR-409-3p, screening a therapeutic agent for hearing loss, and treating hearing loss by administering miR-409-3p. More specifically, the present invention relates to a composition for diagnosing hearing loss, comprising an agent for measuring an expression level of miR-409-3p as an active ingredient; a method for providing information for diagnosing hearing loss; a method for screening a therapeutic agent for hearing loss; and a pharmaceutical composition for preventing or treating hearing loss comprising miR-409-3p as an active ingredient.

### RELATED ART/BACKGROUND

Inflammation is a major area of interest in research on age-related diseases. As tissues age, the body experiences a phenomenon referred to as “chronic inflammation,” also known as “inflammation”, which is a form of inflammation that worsens with advancing age.

Recently, inflammatory pathways and the effects of inflammation on age-related hearing loss (ARHL) have received considerable attention in the field of auditory research. According to recent studies, the cochlea has been shown to be vulnerable to systemic inflammation. Chronic activation of inflammatory cytokines such as IL-1α, IL-2, TNF-α, and NF-κB, which are essential for the initiation, regulation, and amplification of immune responses, can infiltrate inner ear cells, including endolymphatic cells.

Meanwhile, microRNAs (miRNAs) are a class of small, naturally occurring non-coding RNA molecules in organisms. Generally, miRNAs are approximately 20 to 23 nucleotides in length and function by binding to specific target messenger RNA (mRNA) transcripts through complementary base pairing. Interactions between miRNAs and their target mRNA molecules may result in gene silencing, translational repression, or target degradation. It is estimated that miRNAs may target up to 60% of all genes, and each miRNA can influence the expression of hundreds of target genes. Accordingly, as disclosed in Korean Patent Application Publication No. 10-2024-0170692, extensive studies have been conducted on the therapeutic effects of miRNAs in various diseases.

Although such miRNAs are known to affect cell proliferation, differentiation, and growth in the inner ear, the role of miRNAs in the blood of patients with ARHL has not yet been established. Accordingly, the present invention aims to identify biomarkers for age-related hearing loss by analyzing RNA sequencing data from patients with hearing loss, particularly age-related hearing loss, and to contribute to the development of novel approaches for the prevention and treatment of age-related hearing loss.

### SUMMARY

Accordingly, the present inventors have made diligent efforts to develop biomarkers for hearing loss, particularly age-related hearing loss, and as a result, have confirmed that the level of miR-409-3p is reduced in patients with age-related hearing loss. Furthermore, the inventors have identified the potential of the miRNA to contribute to the prevention and treatment of age-related hearing loss, thereby completing the present invention.

Accordingly, an object of the present invention is to provide a composition for diagnosing hearing loss, comprising an agent for measuring the expression level of miR-409-3p as an active ingredient.

Another object of the present invention is to provide a method for providing information for diagnosing hearing loss, comprising:
(a) measuring the expression level of miR-409-3p in a biological sample isolated from a subject; and
(b) comparing the expression level of miR-409-3p measured in step (a) with the expression level of miR-409-3p measured in a biological sample isolated from a normal subject.

Another object of the present invention is to provide a method for screening a therapeutic agent for hearing loss in vitro, comprising:
(a) contacting a cell or tissue expressing miR-409-3p with a candidate therapeutic agent;
(b) measuring an expression level of miR-409-3p in the cells or tissues; and
(c) identifying the candidate therapeutic agent as a therapeutic agent for hearing loss when the expression level of miR-409-3p is increased compared to a control not treated with the candidate therapeutic agent.

Furthermore, still another object of the present invention is to provide a pharmaceutical composition for preventing or treating hearing loss, comprising miR-409-3p as an active ingredient.

In order to solve the above-described problems, the present invention provides a composition for diagnosing hearing loss, comprising an agent for measuring an expression level of miR-409-3p as an active ingredient.

In one embodiment, the composition may further comprise an agent for measuring an expression level of interferon gamma (IFN-γ).

In one embodiment, the agent for measuring the expression level of miR-409-3p may comprise one or more selected from the group consisting of a primer, a probe and an antisense nucleotide that specifically bind to miR-409-3p.

In one embodiment, the hearing loss may be age-related hearing loss.

In one embodiment, the biological sample of the composition may be tissue, cell, whole blood, serum, plasma, saliva, sputum, cerebrospinal fluid or urine isolated from a subject.

In addition, the present invention provides a method for providing information for diagnosing hearing loss, comprising:
(a) measuring an expression level of miR-409-3p in a biological sample isolated from a subject; and
(b) comparing the expression level of miR-409-3p measured in step (a) with the expression level of miR-409-3p measured in a biological sample isolated from a normal subject.

In one embodiment, the method may further comprise:
(c) diagnosing hearing loss when the expression level of miR-409-3p measured in step (a) is lower than the expression level of miR-409-3p measured in a biological sample isolated from a normal subject.

In one embodiment, the method may further measure an expression level of interferon gamma in step (a),
in step (b), the method may further compare the expression level of interferon gamma measured in step (a) with an expression level of interferon gamma measured in a biological sample isolated from a normal subject.

In one embodiment, the expression level may be measured by one or more methods selected from the group consisting of RT-PCR (reverse transcription polymerase chain reaction), quantitative RT-PCR, real-time RT-PCR, Northern blotting and transcriptome analysis.

In one embodiment, the biological sample may include tissue, cell, whole blood, serum, plasma, saliva, sputum, cerebrospinal fluid or urine isolated from a subject.

In one embodiment, the hearing loss may be age-related hearing loss.

In addition, the present invention provides a method for screening a therapeutic agent for hearing loss, comprising:
(a) treating, in vitro, a candidate therapeutic agent for hearing loss to cells or tissues expressing miR-409-3p;
(b) measuring an expression level of miR-409-3p in the cell or tissue; and
(c) identifying the candidate therapeutic agent as a therapeutic agent for hearing loss when the expression level of miR-409-3p is increased compared to a control not treated with the candidate therapeutic agent.

Furthermore, the present invention provides a pharmaceutical composition for preventing or treating hearing loss, comprising miR-409-3p as an active ingredient.

In one embodiment, the miR-409-3p may comprise a nucleotide sequence of SEQ ID NO: 1.

In one embodiment, the miR-409-3p may be provided in a form included in a vector or introduced into a cell.

In one embodiment, the miR-409-3p may reduce a level of interferon gamma and/or p16.

In the present invention, it was confirmed that patients with hearing loss exhibit reduced expression levels of miR-409-3p, and in particular, such reduction was observed in patients with age-related hearing loss. In addition, regulation of interferon gamma (IFNG) and p16 signaling by miR-409-3p was confirmed in blood samples from patients with hearing loss. Specifically, it was confirmed that overexpression of miR-409-3p reduces the expression of IFNG and p16, which is an aging-related marker, thereby demonstrating that the present invention can contribute to a novel approach for the prevention and treatment of age-related hearing loss. Accordingly, the biomarkers and the pharmaceutical composition for treating hearing loss according to the present invention have high utility and applicability.

### BRIEF DESCRIPTION OF THE DRAWINGS

A of FIG. 1 is a heatmap showing differential expression of mRNA levels in patients with age-related hearing loss, B of FIG. 1 is a Venn diagram showing the numbers of genes that are upregulated or downregulated in patients with age-related hearing loss based on mRNA analysis, C of FIG. 1 is a volcano plot showing expression levels of candidate genes for hearing loss biomarkers, D of FIG. 1 is a scatter plot showing expression levels of candidate genes for hearing loss biomarkers.

FIG. 2 shows results of validating mRNA expression levels of candidate genes (ADAM12, FLT1, TNFRSF25, IFNG, TNFAIP6) using RT-qPCR.

FIG. 3 shows results of analyzing expression levels of IFNG and a target miRNA (miR-409-3p) in a normal hearing group and ARHL group.

A of FIG. 4 shows a target site of miR-409-3p within the IFNG gene, B of FIG. 4 shows results of evaluating luciferase activity by co-expressing a miR-409-3p vector and a 3′ UTR of an IFNG reporter.

FIG. 5 shows results of analyzing changes in expression levels of IFNG and p16 by Western blot after transfection with miR-409-3p at various concentrations (500, 1000, 2000 ng).

### DETAILED DESCRIPTION

Hereinafter, the present invention will be described in more detail.

Meanwhile, each description and embodiment disclosed herein may be applied to other descriptions and embodiments. That is, all combinations of the various elements disclosed herein fall within the scope of the present invention. In addition, the scope of the present invention should not be construed as being limited by the specific descriptions set forth below.

In addition, a person having ordinary skill in the art may recognize or confirm a number of equivalents of specific aspects of the present invention described in the present application using only routine experimentation. Such equivalents are also intended to be included in the present invention.

As described above, the present inventors have confirmed that hearing loss can be diagnosed by examining the level of miR-409-3p.

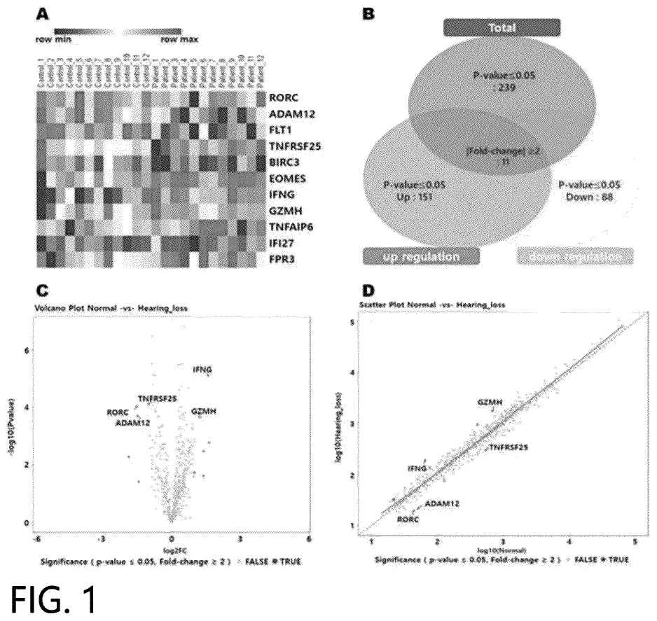

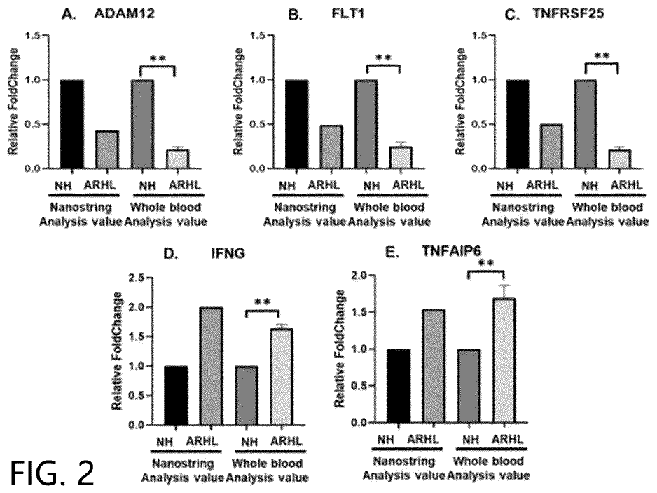

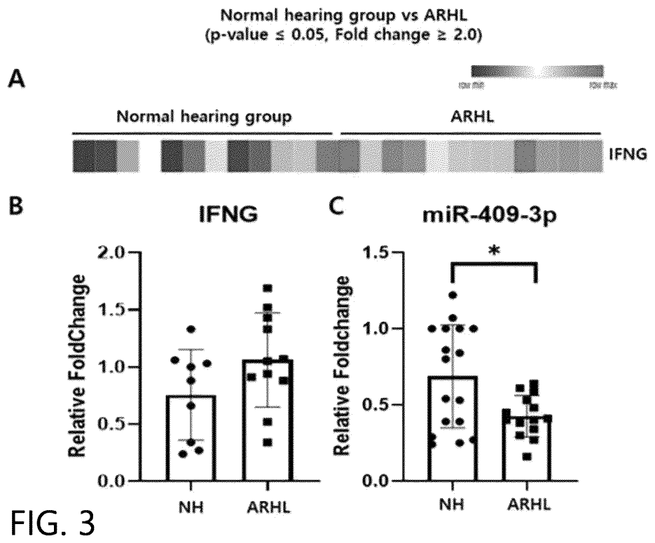

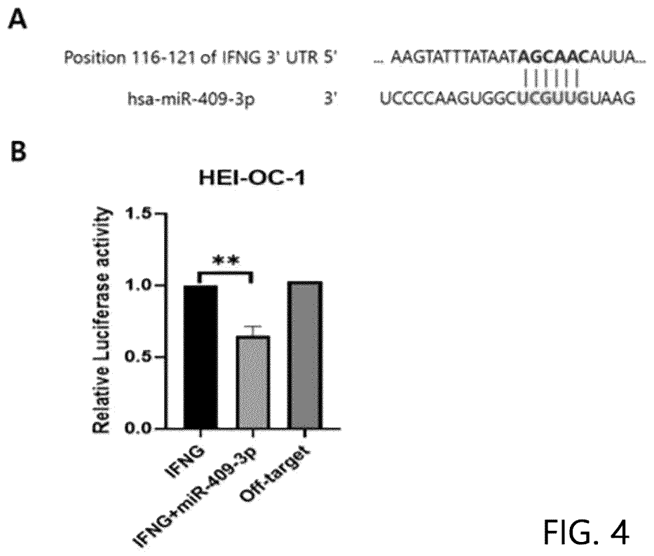

In one specific embodiment of the present invention, as shown in FIG. 1, differential expression of mRNA was analyzed in hearing loss patients. As shown in FIG. 2, it was confirmed that the genes ADAM12, FLT1, TNFRSF25, IFNG and TNFAIP6 exhibit differential expression in hearing loss patients, and that miR-409-3p is downregulated in hearing loss patients (FIG. 3).

Accordingly, the first aspect of the present invention relates to a composition for diagnosing hearing loss, comprising an agent for measuring an expression level of miR-409-3p as an active ingredient.

As used herein, the term “miR” or “micro RNA” refers to a non-coding RNA consisting of 21 to 23 nucleotides that regulates gene expression post-transcriptionally by promoting degradation of target RNA or by inhibiting their translation.

In general, microRNAs are transcribed as primary microRNAs (pri-miRNAs), which are precursor transcripts that may include one or more hairpin structures. Such pri-miRNAs are processed by enzymatic action into precursor microRNAs (pre-miRNAs) having a hairpin structure with a length of about 70 to 80 nucleotides. Subsequently, the pre-miRNAs are transported out of the nucleus and, in the cytoplasm, the hairpin structure is cleaved by an RNase (Dicer). During this process, Dicer binds to the 3′ end of the hairpin, cleaves the loop connecting the 3′ and 5′ arms, and generates an unstable double-stranded miRNA, which ultimately gives rise to mature miR-3p and miR-5p.

In the present invention, the miR-409-3p is derived from the 3p end of the pre-miRNA miR-409 having a hairpin structure, as described above. The present inventors have particularly discovered that miR-409-3p is downregulated in patients with hearing loss. The miR-409-3p biomarker according to the present application includes not only mature miR-409-3p but also a precursor (pre-miR-409) capable of producing the same.

As used herein, the term “biomarker” or “diagnosis marker” refers to a substance capable of diagnosing hearing loss by distinguishing a sample derived from a patient with hearing loss from a control sample, and includes a non-coding nucleic acid that exhibits a reduced expression level in a sample derived from a patient with hearing loss including a patient having a higher severity of hearing loss, as compared with a control.

In the present invention, the biomarker may be detected at the level of detection of the presence or absence of miRNA and/or its expression level itself, changes in expression level or differences in expression level through quantitative and/or qualitative analysis, and may be used for the diagnosis of hearing loss.

In a specific embodiment of the present invention, target miRNAs for IFNG were searched. As a result, a binding target site of miR-409-3p was identified in the 3′ UTR of IFNG (A of FIG. 4). After co-transfection of a miR-409-3p vector and a 3′ UTR of IFNG reporter, a significant decrease in luciferase activity was confirmed (B of FIG. 4), thereby confirming that IFNG is a direct target of miR-409-3p.

Accordingly, in the present invention, the composition may further comprise an agent for measuring an expression level of interferon gamma (IFN-γ).

In the present invention, the agent for measuring the expression level is a material capable of detecting the presence or absence of miRNA or its CRNA or cDNA and/or detecting the expression level itself, changes in expression level, or differences in expression level. The composition may further comprise, together with the agent for measuring the expression level, reagents suitable for detecting the biomarker.

In the present invention, the agent for measuring the expression level of miR-409-3p may comprise one or more selected from the group consisting of a primer, a probe and an antisense nucleotide that specifically bind to miR-409-3p, but is not limited thereto.

In the present invention, the primer, probe or antisense nucleotide may comprise a nucleotide sequence complementary to miR-409-3p. As used herein, the term “complementary” refers to having a degree of complementarity sufficient to selectively hybridize to the nucleotide sequence of miR-409-3p of the present invention under certain hybridization or annealing conditions. Accordingly, the term “complementary” has a meaning different from “perfectly complementary,” and the primer, probe or antisense nucleotide of the present invention may contain one or more mismatched nucleotide sequences, provided that it is capable of selectively hybridizing to the nucleotide sequence of miR-409-3p.

As used herein, the term “probe” refers to a linear oligomer of natural or modified monomers or linkages, including deoxyribonucleotides and ribonucleotides, which is capable of specifically hybridizing to a target nucleotide sequence and may be naturally occurring or artificially synthesized. The probe may include naturally occurring dNMPs (i.e., dAMP, dGMP, dCMP and dTMP), nucleotide analogs or derivatives thereof. In addition, the probe may also include ribonucleotides. For example, the probe may include backbone-modified nucleotides such as peptide nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate nucleic acid; sugar-modified nucleotides such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA and anhydrohexitol DNA; and base-modified nucleotides such as C-5-substituted pyrimidines (wherein the substituents include fluoro-, bromo-, chloro-, iodo-, methyl-, ethyl-, vinyl-, formyl-, ethynyl-, propynyl-, alkynyl-, thiazolyl-, imidazolyl- and pyridyl-), 7-deazapurines having C-7 substituents (wherein the substituents include fluoro-, bromo-, chloro-, iodo-, methyl-, ethyl-, vinyl-, formyl-, alkynyl-, alkenyl-, thiazolyl-, imidazolyl- and pyridyl-), inosine, and diaminopurine. Those skilled in the art will be able to select an appropriate probe sequence according to the present invention with reference to the sequence of miR-409-3p disclosed herein.

As used herein, the term “primer” refers to a single-stranded oligonucleotide that can serve as a starting point for template-directed DNA synthesis under suitable conditions (i.e., four different nucleoside triphosphates and a polymerization enzyme) in an appropriate buffer, at an appropriate temperature. The appropriate length of the primer may vary depending on various factors, such as temperature and the intended use of the primer, but is typically about 15 to 30 nucleotides. The sequence of the primer does not need to be perfectly complementary to a portion of the template sequence, and it is sufficient that the primer has a degree of complementarity adequate to hybridize with the template and perform its inherent primer function. Accordingly, the primer does not need to have a sequence perfectly complementary to the nucleotide sequence of the template miR-409-3p, and it is sufficient that the primer has sufficient complementarity to hybridize to the sequence and function as a primer. Those skilled in the art will be able to select an appropriate primer sequence according to the present invention with reference to the sequence of miR-409-3p disclosed herein.

In addition, the primer may be used in a gene amplification reaction. The amplification reaction refers to a reaction for amplifying a nucleic acid molecule. Various amplification reactions have been reported in the art, including, but not limited to, polymerase chain reaction (PCR), reverse transcription polymerase chain reaction (RT-PCR), real-time polymerase chain reaction (real-time PCR), ligase chain reaction (LCR), repair chain reaction, transcription-mediated amplification (TMA), self-sustained sequence replication, selective amplification of target polynucleotide sequences, consensus sequence-primed polymerase chain reaction, arbitrarily primed polymerase chain reaction, nucleic acid sequence-based amplification (NASBA), strand displacement amplification (SDA) and loop-mediated isothermal amplification (LAMP).

As used herein, the term “antisense nucleotide” should be understood to mean a nucleotide having a sequence complementary to another sequence, particularly a sequence complementary to miR-409-3p. Targeting miR-409-3p may also be understood to include targeting downstream targets of miR-409-3p. Importantly, inhibition of miR-409-3p, for example through a nucleotide having at least a complementary sequence to miR-409-3p, may induce derepression or even overexpression of targets of miR-409-3p, such as IFNG.

As used herein, the term “hearing loss” refers to any condition in which hearing is reduced or lost.

In the present invention, the hearing loss may be age-related hearing loss.

As used herein, the term “age-related hearing loss” or “presbycusis” refers to hearing loss associated with aging, which occurs as part of normal aging and results from degeneration of sensory receptor cells in the spiral organ of Corti in the inner ear. Other causes may include loss of flexibility of the basilar membrane of the cochlea, as well as a reduction in the number of nerve fibers of the vestibulocochlear nerve. Currently, there are no known treatments for presbycusis or permanent hearing impairment caused by excessive noise exposure.

In the present invention, a biological sample of the composition may be a tissue, cell, whole blood, serum, plasma, saliva, sputum, cerebrospinal fluid or urine isolated from a subject.

As used herein, the term “diagnosis” includes determining a subject's susceptibility to a specific disorder, determining whether a subject currently has a specific disease or disorder, determining the prognosis of a subject afflicted with a specific disease or disorder, determining recurrence after treatment of a disease or performing therametrics (e.g., monitoring the status of a subject to provide information on therapeutic efficacy).

As used herein, the term “composition for diagnosing” refers to an integrated mixture or device comprising means for measuring the expression level of miR-409-3p in order to determine whether a subject has developed hearing loss or to predict the likelihood of developing hearing loss, and may also be referred to as a “kit for diagnosing”. Since the composition for diagnosing of the present invention includes means for measuring the biomarker identified in the present invention, the term “composition for diagnosing” may also be expressed as a “quantification device” for the biomarker.

In addition, a second aspect of the present invention relates to a kit for diagnosing hearing loss, comprising an agent for measuring an expression level of miR-409-3p.

In the present invention, the kit may be an RT-PCR kit, a competitive RT-PCR kit, a real-time RT-PCR kit, a digital PCR kit, a DNA chip kit, a protein chip kit or an immunoassay kit.

The kit of the present invention may comprise the agent for measuring the expression level of miR-409-3p, as well as a composition, a solution or a device comprising one or more other components suitable for an analysis method.

In a specific embodiment, the kit may be a diagnostic kit which is characterized in that it comprises essential elements necessary to perform reverse transcription polymerase reaction. The reverse transcription polymerase reaction kit comprises each primer pair specific for the gene encoding biomarker. In addition, a primer specific to a nucleic acid sequence of a control gene may be included. Other reverse transcription polymerase reaction kit may include test tubes or other suitable containers, reaction buffers (with varying pH and magnesium concentrations), deoxynucleotides (dNTPs), enzymes such as Taq polymerase and reverse transcriptase, DNase, RNase inhibitors, DEPC water, sterile water and the like.

In still another embodiment, it may be a diagnostic kit, which is characterized in that it includes essential elements necessary to perform the DNA chip. The DNA chip kit may include a substrate to which cDNA or oligonucleotide corresponding to a gene or fragment thereof is attached, and reagents, agents, enzymes an the like for constructing a fluorescently labeled probes. In addition the substrate may include cDNA or oligonucleotide corresponding to a control gene or fragment thereof.

In the present invention, the kit may further comprise an agent for measuring an expression level of interferon gamma.

In addition, a third aspect of the present invention relates to a method for diagnosing and treating hearing loss, comprising:
(a) measuring an expression level of miR-409-3p in a biological sample isolated from a subject; and
(b) comparing the expression level of miR-409-3p measured in step (a) with an expression level of miR-409-3p measured in a biological sample isolated from a normal subject.

In the present invention, the method may further comprise (c) diagnosing hearing loss when the expression level of miR-409-3p measured in step (a) is lower than the expression level of miR-409-3p measured in a biological sample isolated from the normal subject.

In the present invention, the expression level being lower than that of a normal subject means that the level of miR-409-3p is significantly lower than that of a normal subject without hearing loss. Specifically, this means a decrease of about 10% or more, about 20% or more, about 30% or more, about 40% or more, or about 50% or more compared to the normal subject, but does not exclude ranges outside these values.

In the present invention, the method may further comprise
(d) applying therapy for hearing loss to the subject of step (a).

In the present invention, the therapy for hearing loss of step (d) may be one or more drug therapies selected from the group consisting of steroids, vasodilators, antioxidants, diuretics, immunosuppressant and interferon gamma inhibitors.

In the present invention, an expression level of interferon gamma may be further measured in step (a),
and the expression level of interferon gamma measured in step (a) may be further compared with an expression level of interferon gamma measured in a biological sample isolated from a normal subject in step (b).

In the present invention, when the expression level of interferon gamma is additionally measured in step (a), the method may further comprise:
(c) diagnosing hearing loss when the expression level of miR-409-3p measured in step (a) is lower than the expression level of miR-409-3p measured in the biological sample isolated from the normal subject, and the expression level of interferon gamma measured in step (a) is higher than the expression level of interferon gamma measured in the biological sample isolated from the normal subject.

In the present invention, the expression level being higher than that of a normal subject means that the level of interferon gamma is significantly higher than that of a normal subject without hearing loss. Specifically, this means an increase of about 10% or more, about 20% or more, about 30% or more, about 40% or more, or about 50% or more compared to the normal subject, but does not exclude ranges outside these values.

In the present invention, the method may further comprise (d) applying therapy for hearing loss to the subject of step (a).

In the present invention, the expression level may be measured by one or more methods selected from the group consisting of RT-PCR (reverse transcription polymerase chain reaction), quantitative RT-PCR, real-time RT-PCR, Northern blotting and transcriptome analysis.

In the present invention, the biological sample may include a tissue, cell, whole blood, serum, plasma, saliva, sputum, cerebrospinal fluid or urine isolated from a subject.

In the present invention, the hearing loss may be age-related hearing loss.

In addition, a fourth aspect of the present invention relates to a method for providing information required for determining a therapy for hearing loss, comprising:
(a) measuring an expression level of miR-409-3p in a biological sample isolated from a subject; and
(b) comparing the expression level of miR-409-3p measured in step (a) with an expression level of miR-409-3p measured in a biological sample isolated from a normal subject.

In the present invention, the method may further comprise (c) diagnosing hearing loss when the expression level of miR-409-3p measured in step (a) is lower than the expression level of miR-409-3p measured in a biological sample isolated from the normal subject.

In the present invention, the method may further comprise (d) applying therapy for hearing loss to the subject of step (a).

In the present invention, the therapy for hearing loss of step (d) may be one or more drug therapies selected from the group consisting of steroids, vasodilators, antioxidants, diuretics, immunosuppressant and interferon gamma inhibitors.

In the present invention, the interferon gamma inhibitor may be a direct interferon gamma inhibitor such as emapalumab, or an inhibitor of the interferon gamma receptor or its signal transduction, such as a JAK inhibitor (e.g., ruxolitinib, tofacitinib, baricitinib) or a STAT signaling inhibitor.

In the present invention, an expression level of interferon gamma may be further measured in step (a),
and the expression level of interferon gamma measured in step (a) may be further compared with an expression level of interferon gamma measured in a biological sample isolated from a normal subject in step (b).

In the present invention, the method may further comprise (c) diagnosing hearing loss when the expression level of miR-409-3p measured in step (a) is lower than the expression level of miR-409-3p measured in the biological sample isolated from the normal subject, and the expression level of interferon gamma measured in step (a) is higher than the expression level of interferon gamma measured in the biological sample isolated from the normal subject.

In the present invention, the method may further comprise (d) applying therapy for hearing loss to the subject of step (a).

In addition, a fifth aspect of the present invention relates to a method for screening a therapeutic agent for hearing loss in vitro, comprising:
(a) contacting a cell or tissue expressing miR-409-3p with a candidate therapeutic agent;
(b) measuring an expression level of miR-409-3p in the cells or tissue; and
(c) identifying the candidate therapeutic agent as a therapeutic agent for hearing loss when the expression level of miR-409-3p is increased compared to a control not treated with the candidate therapeutic agent.

In the present invention, the cells may be auditory cells and the tissue may be cochlear tissue, but are not limited thereto.

In the present invention, the candidate therapeutic agent may be a peptide, protein, non-peptidic compound, active compound, fermentation product, cell extract, plant extract or animal tissue extract, but is not limited thereto.

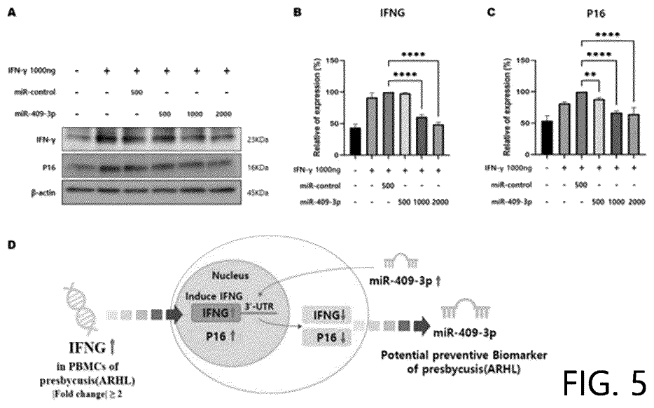

In a specific embodiment of the present invention, a significant decrease in luciferase activity was confirmed after co-transfection of a miR-409-3p vector and a 3′UTR of IFNG reporter (B of FIG. 4). In another specific embodiment of the present invention, it was confirmed that miR-409-3p downregulated the expression of IFNG and p16 (FIG. 5), thereby demonstrating that miR-409-3p exhibits a therapeutic effect on hearing loss.

Accordingly, a sixth aspect of the present invention relates to a pharmaceutical composition for preventing or treating hearing loss, comprising miR-409-3p as an active ingredient.

In the present invention, the hearing loss may be age-related hearing loss.

In the present invention, the miR-409-3p may comprise a nucleotide sequence of SEQ ID NO: 1, and the nucleotide sequence of SEQ ID NO: 1 is as shown in Table 1 below.

TABLE 1

| Sequence | | SEQ ID |
|---|---|---|
| Name | Sequence (5′-3′) | NO. |
| miR-409-3p | GAATGTTGCTCGGTGAACCCCT | 1 |

In the present invention, the miR-409-3p may be provided in a form included in a vector or introduced into a cell. In the present invention, the miR-409-3p may reduce the level of interferon gamma and/or p16.

As used herein, the term “prevention” refers to any action by which hearing loss or related diseases are suppressed or the onset thereof is delayed by the composition of the present invention.

As used herein, the term “treatment” refers to any action by which parameters associated with hearing loss or related diseases, for example, the severity of symptoms, are improved or ameliorated by the composition of the present invention.

The pharmaceutical composition of the present invention may be formulated in various dosage forms for oral or parenteral administration. When formulating the composition, one or more buffers (e.g., saline or phosphate-buffered saline (PBS)), antioxidants, bacteriostatic agents, chelating agents (e.g., EDTA or glutathione), fillers, extenders, binders, adjuvants (e.g., aluminum hydroxide), suspending agents, thickening agents, wetting agents, disintegrants, surfactants, diluents or excipients may be used.

Solid preparations for oral administration include tablets, pills, powders, granules, capsules, and the like. Such solid preparations may be prepared by mixing one or more compounds with at least one excipient, for example, starches (including corn starch, wheat starch, rice starch, potato starch, and the like), calcium carbonate, sucrose, lactose, dextrose, sorbitol, mannitol, xylitol, erythritol, maltitol, cellulose, methylcellulose, sodium carboxymethylcellulose, hydroxypropyl methylcellulose or gelatin. For example, tablets or coated tablets may be obtained by blending an active ingredient with a solid excipient, pulverizing the mixture, adding suitable auxiliaries, and processing the resulting mixture into granules.

In addition to simple excipients, lubricants such as magnesium stearate and talc may also be used. Liquid preparations for oral administration include suspensions, oral solutions, emulsions, or syrups, which may contain, in addition to commonly used simple diluents such as water or liquid paraffin, various excipients including wetting agents, sweeteners, flavoring agents or preservatives. In some cases, crosslinked polyvinylpyrrolidone, agar, alginic acid or sodium alginate may be added as disintegrants, and the composition may further comprise anti-caking agents, fragrances, emulsifiers, solubilizers, dispersants, flavoring agents, antioxidants, coating agents, pigments or preservatives.

Preparations for parenteral administration include sterile aqueous solutions, non-aqueous solvents, suspending agents, emulsions, lyophilized formulations or suppositories. Examples of non-aqueous solvents and suspending agents that may be used include propylene glycol, polyethylene glycol, vegetable oils such as olive oil and injectable esters such as ethyl oleate. Bases for suppositories may include witepsol, macrogol, tween 61, cocoa butter, laurin fat, glycerol and gelatin.

The composition of the present invention may be administered orally or parenterally, and for parenteral administration, the composition may be formulated in the form of injectable preparations for intraperitoneal, rectal, intravenous, intramuscular or subcutaneous administration according to methods well known in the art.

The injectable preparations must be sterile and protected from contamination by microorganisms such as bacteria and fungi. Suitable carriers for injectable preparations include, but are not limited to, solvents or dispersion media comprising water, ethanol, polyols (e.g., glycerol, propylene glycol and liquid polyethylene glycol), mixtures thereof and/or vegetable oils. More preferably, suitable carriers may include isotonic solutions such as Hanks' solution, Ringer's solution, PBS containing triethanolamine, sterile water for injection, 10% ethanol, 40% propylene glycol or 5% dextrose. In order to protect the injectable preparations from microbial contamination, various antibacterial and antifungal agents such as parabens, chlorobutanol, phenol, sorbic acid, and thimerosal may be additionally included. In most cases, the injectable preparations may further comprise isotonic agents such as sugars or sodium chloride.

The composition of the present invention is administered in a pharmaceutically effective amount. As used herein, the term “pharmaceutically effective amount” refers to an amount sufficient to treat a disease at a reasonable benefit/risk ratio applicable to medical treatment. The effective dosage level may be determined depending on factors including type and severity of the disease in a subject, activity of the drug, sensitivity to the drug, time of administration, route of administration and rate of excretion, duration of treatment, drugs used in combination and other factors well known in the medical field. The composition of the present invention may be administered as a single therapeutic agent or in combination with other therapeutic agents, and may be administered sequentially or simultaneously with conventional therapeutic agents, and may be administered in a single dose or in multiple doses. That is, the total effective amount of the composition of the present invention may be administered to a subject as a single dose, or may be administered by a fractionated treatment protocol in which multiple doses are administered over an extended period. It is important to administer an amount that achieves the maximum effect with the minimum amount without causing side effects, taking all of the above factors into consideration, and such an amount may be readily determined by a person skilled in the art.

The dosage of the pharmaceutical composition of the present invention may vary depending on the body weight, age, sex, health condition, diet, time of administration, method of administration, rate of excretion and severity of the disease of the subject.

The composition of the present invention may be used alone, or in combination with methods using surgery, radiation therapy, hormone therapy, chemotherapy and biological response modifiers.

Furthermore, a seventh aspect of the present invention relates to a method for preventing or treating hearing loss, comprising administering a pharmaceutical composition comprising miR-409-3p to a subject in need thereof.

In the present invention, the miR-409-3p may comprise a nucleotide sequence of SEQ ID NO: 1.

In the present invention, the miR-409-3p may be provided in a form included in a vector or introduced into a cell.

In the present invention, the miR-409-3p may reduce a level of interferon gamma and/or p16.

In one embodiment, the pharmaceutical composition may be administered into the middle ear cavity through the tympanic membrane and subsequently delivered to the inner ear via the round window.

In another embodiment, the pharmaceutical composition may be directly administered to the inner ear using microinjection, a catheter, a drug delivery device or a biodegradable carrier.

In still another embodiment, the pharmaceutical composition may be administered in a form encapsulated in nanoparticles, liposomes, polymeric carriers, viral vectors, or non-viral delivery systems, thereby improving the stability, bioavailability or delivery efficiency of miR-409-3p to the target tissue.

Hereinafter, the present invention will be described in more detail with reference to the following Examples. However, the following Examples are provided for illustrative purposes only and are not intended to limit the scope of the present invention.

### Example 1

1. Confirmation of Decreased miR-409-3p Levels in Patients with Hearing Loss
1-1. Patients with Age-Related Hearing Loss

Hearing in both ears of all enrolled participants was measured by pure-tone audiometry using an Interacoustics AC40 (Denmark) clinical audiometer in accordance with the manufacturer's instructions. The inclusion criteria for the Normal Hearing (NH) group were defined as an average hearing threshold of ≤25 dB at 250 Hz, 500 Hz, 1000 Hz, 2000 Hz and 4000 Hz, and the Age-Related Hearing Loss (ARHL) group was defined as an average hearing threshold of ≥40 dB at 250 Hz, 500 Hz, 1000 Hz, 2000 Hz and 4000 Hz. Patients with a history of acute or chronic infection or symptoms of infection, hypertension, diabetes mellitus, chronic kidney disease, or other otologic diseases or surgery were excluded from the subject population.

1-2. Isolation of Peripheral Blood Mononuclear Cells (PBMCs) from Blood and mRNA Analysis

Blood samples were collected from 12 subjects in a Normal Hearing group (28 #12.89 years, male:female=5:7) and 12 subjects in an ARHL group (68±8.56 years, male:female=7:5), and RNA was extracted using a QIAamp RNA Blood Mini Kit (Qiagen, Germany) and subsequently used for RT-PCR analysis. Blood collected in EDTA tubes was subjected to Ficoll-Paque density gradient centrifugation to isolate a peripheral blood mononuclear cell (PBMC) layer, and RNA was extracted from the PBMCs isolated as described above. The RNA samples were analyzed using a NanoString nCounter Analytic System (NanoString Technologies, Inc., WA, USA).

### 1-3. Quantification of Gene Expression and Analysis of Differentially Expressed Genes

Gene expression levels were quantified using the geNorm algorithm of nCounter Advanced Analysis ver. 2.0.115, and differentially expressed gene expression levels were analyzed.

### 1-4. Verification of Target Gene Expression

To verify the expression of candidate genes identified from the mRNA analysis results, RT-qPCR was performed using blood samples from the Normal Hearing group and the ARHL group. Reverse transcription was carried out using a miScript II RT Kit (Qiagen, Germany), and RT-qPCR was performed using SYBR Green on an ABI StepOnePlus system. The primer information used for analysis is shown in Table 2 below.

TABLE 2

| Sequence | | SEQ ID |
|---|---|---|
| Name | Sequence (5′-3′) | NO. |
| miR-409-3p | GAATGTTGCTCGGTGAACCCCT | 2 |
| primer | | |
| IFN primer | GGGGCTCAGTTTCCTCATCT | 3 |
| FW | | |
| IFN primer | TAGAGACTTGCAGTGGGGTG | 4 |
| RV | | |

Using NanoString mRNA analysis data obtained from PBMCs of the ARHL group, expression of target genes was analyzed. As a result, as shown in FIG. 1, 11 mRNAs showing increased or decreased expression were identified in the ARHL group.

To verify the mRNA analysis results of the differentially expressed genes (DEG), RT-qPCR was performed using blood samples. As shown in FIG. 2, five genes (ADAM12, FLT1, TNFRSF25, IFNG and TNFAIP6) showing a correlation with ARHL were identified. In particular, the expression level of the IFNG gene was higher in the ARHL group than in the normal hearing group, showing a statistically significant difference. In addition, the expression of miR-409-3p was found to be downregulated in the ARHL group in an inverse correlation with interferon gamma (IFN-γ) (IFNG), as shown in FIG. 3.

### Example 2

2. Identification of Target Genes of miR-409-3p
2-1. Gene Ontology Analysis of miRNAs Associated with IFNG

In order to identify the functions of the DEGs, KEGG analysis and gene ontology analysis were performed using the EnrichR database.

2-2. Transfection of IFNG 3′ UTR and miRNA Vectors into Auditory Cells

House Ear Institute-Organ of Corti 1 (HEI-OC-1) cells were transfected with an IFNG 3′ UTR clone (Origene, USA) and miRNA-409-3p (Genecopoeia, USA).

To identify potential regulators of IFNG expression in ARHL, bioinformatics analysis was performed. Target miRNAs for IFNG were searched using databases such as miRTarBase and miRBase. As a result, as shown in A of FIG. 4, a binding target site for miR-409-3p was observed in the 3′ untranslated region (3′ UTR) of IFNG. In addition, after co-transfection of a miR-409-3p vector and an IFNG 3′ UTR reporter, a significant decrease in luciferase activity was confirmed (B of FIG. 4), thereby demonstrating that IFNG is a direct target of miR-409-3p in auditory cells (HEI-OC-1).

### Example 3

3. Confirmation of Regulation of IFNG and/or p16 Expression by miR-409-3p

Cells were lysed using RIPA buffer composed of 25 mM Tris-HCl (pH 7.6), 150 mM NaCl, 1% NP-40, 1% sodium deoxycholate and 0.1% SDS, and protein was quantified using a Pierce BCA Protein Assay Kit (Thermo Fisher Scientific Inc.). Equal amounts of protein were loaded onto 4-15% SDS polyacrylamide precast gels (Bio-Rad Laboratories Inc.), electrophoresed at 70 V for 30-40 minutes, and then further electrophoresed at 110 V for 50-60 minutes. The separated proteins were transferred onto PVDF membranes, blocked with 5% BSA at room temperature for 1 hour, and then incubated overnight at 4° C. with primary antibodies against IFN-γ and p16 (1:1000 dilution, Cell Signaling Technology, Danvers, MA, USA) and β-actin (1:5000 dilution, Cell Signaling Technology, USA). The membranes were then incubated with horseradish peroxidase (HRP)-conjugated secondary antibodies (1:3000 dilution, Cell Signaling Technology, USA) at room temperature for 2 hours. Protein signals were detected using Super Signal West Atto Ultimate Sensitivity Chemiluminescent Substrate (Thermo Fisher).

As a result of western blot analysis, it was confirmed, as in Example 2, that miR-409-3p regulates the expression of the IFNG gene (FIG. 5). Furthermore, when auditory cells were co-transfected with the IFNG gene and various concentrations (500, 1000, 2000 ng) of miR-409-3p, the expression of IFNG decreased as the concentration of miR-409-3p increased (A and B of FIG. 5). That is, overexpression of miR-409-3p was confirmed to downregulate IFNG. In addition, detection of the aging-related marker p16 demonstrated that the expression of p16 protein decreased as the concentration of miR-409-3p increased (A and C of FIG. 5). Accordingly, it was confirmed that miR-409-3p binding to the 3′ UTR of IFNG induces downregulation of IFNG and p16 signaling pathways. Therefore, the present invention proposes miR-409-3p as a biomarker and a potential therapeutic agent for ARHL.

What is claimed is:

1. A method for diagnosing and treating hearing loss, comprising:
(a) measuring an expression level of miR-409-3p in a biological sample isolated from a subject; and
(b) comparing the expression level of miR-409-3p measured in step (a) with an expression level of miR-409-3p measured in a biological sample isolated from a normal subject.

2. The method of claim 1, further comprising (c) diagnosing hearing loss when the expression level of miR-409-3p measured in step (a) is lower than the expression level of miR-409-3p measured in a biological sample isolated from the normal subject.

3. The method of claim 2, further comprising (d) applying therapy for hearing loss to the subject of step (a).

4. The method of claim 3, wherein the therapy for hearing loss of step (d) is one or more drug therapies selected from the group consisting of steroids, vasodilators, antioxidants, diuretics, immunosuppressant and interferon gamma inhibitors.

5. The method of claim 1, wherein an expression level of interferon gamma is further measured in step (a),
and wherein the expression level of interferon gamma measured in step (a) is further compared with an expression level of interferon gamma measured in a biological sample isolated from a normal subject in step (b).

6. The method of claim 5, further comprising (c) diagnosing hearing loss when the expression level of miR-409-3p measured in step (a) is lower than the expression level of miR-409-3p measured in the biological sample isolated from the normal subject, and the expression level of interferon gamma measured in step (a) is higher than the expression level of interferon gamma measured in the biological sample isolated from the normal subject.

7. The method of claim 6, further comprising (d) applying therapy for hearing loss to the subject of step (a).

8. The method of claim 1, wherein the expression level is measured by one or more methods selected from the group consisting of RT-PCR (Reverse transcription polymerase chain reaction), quantitative RT-PCR, real-time RT-PCR, Northern blotting and transcriptome analysis.

9. The method of claim 1, wherein the biological sample includes a tissue, cell, whole blood, serum, plasma, saliva, sputum, cerebrospinal fluid or urine isolated from a subject.

10. The method of claim 1, wherein the hearing loss is age-related hearing loss.

11. A method for screening a therapeutic agent for hearing loss in vitro, comprising:
(a) contacting a cell or tissue expressing miR-409-3p with a candidate therapeutic agent;
(b) measuring an expression level of miR-409-3p in the cell or tissue; and
(c) identifying the candidate therapeutic agent as a therapeutic agent for hearing loss when the expression level of miR-409-3p is increased compared to a control not treated with the candidate therapeutic agent.

12. A method for preventing or treating hearing loss, comprising administering a pharmaceutical composition comprising miR-409-3p to a subject in need thereof.

13. The method of claim 12, wherein the miR-409-3p comprises a nucleotide sequence of SEQ ID NO: 1.

14. The method of claim 12, wherein the miR-409-3p is provided in a form included in a vector or introduced into a cell.

15. The method of claim 12, wherein the miR-409-3p reduces a level of interferon gamma and/or p16.
